(19) United States
(12) Patent Application Publication
(10) Pub. No.: US 2025/0312142 A1
(43) Pub. Date: Oct. 9, 2025

(54) MEDICAL MESH ASSEMBLY

(71) Applicant: Sangjoon YOON, Seoul (KR)

(72) Inventor: Sangjoon YOON, Seoul (KR)

(21) Appl. No.: 19/096,762

(22) Filed: Apr. 1, 2025

(30) Foreign Application Priority Data
Apr. 8, 2024 (KR) 10-2024-0047279

(51) Int. Cl. A61F 2/10; A61F 2/00

(52) U.S. Cl. CPC A61F 2/105; A61B 17/06166; A61F 2/0063; A61B 2017/06176; A61F 2002/0068; A61F 2230/0069

(57) ABSTRACT

According to an embodiment of the present disclosure, a mesh assembly is provided that exhibits enhanced fixing and pulling forces with respect to skin tissue, and provides excellent adhesion, retention, and fixation to the skin tissue. The mesh assembly according to an embodiment of the present disclosure is configured to be inserted into skin tissue of a human body and may include a mesh member formed in a cylindrical shape with a plurality of wire members crossing each other.

### CROSS REFERENCE TO RELATED APPLICATION

The present application claims priority to Korean Patent Application No. 10-2024-0047279, Apr. 8, 2024 (M-D-Y) the entire contents of which are incorporated herein for all purposes by this reference.

### BACKGROUND

### Technical Field

The present disclosure relates to a medical instrument and, in more detail, to a mesh assembly for cosmetic procedures and plastic procedures.

### Description of the Related Art

Wrinkles are an important matter relating to aging and aging of the outward appearance may be more clearly recognized due to wrinkles. Accordingly, procedures of effectively removing wrinkles and recovering the elasticity of the skin are generally used in the field of skin care

Botox injection and filler injection are used as wrinkle removal procedures. Further, there are procedures of inserting a thread into the skin to lift up the skin and increase the elasticity. In the procedures using a thread, a thread is inserted into the skin using a cannula or a needle. A thread is fixed in the tissues inside the skin, thereby serving to reduce wrinkles and lift up the skin. Various types of threads are used in the procedures using threads and there is a need for a new type of skin insertion structure having increased fixing force and pulling force.

### SUMMARY

According to an aspect of the present disclosure, there is provided a mesh assembly configured to be inserted into the skin tissue of a human body.

The mesh assembly may include a mesh member comprising a plurality of wire members crossing each other. The mesh member may be configured in a cylindrical shape to define a passage having a predetermined diameter therein, and one or more of the wire members may include at least one protrusion extending from its surface. The passage defined inside the mesh member may be an empty space in which no other material is disposed.

In certain embodiments, the extension directions of the protrusions may differ between a first section and a second section of the wire member. In a more specific embodiment, the protrusions in the first section may extend in a direction opposite to that of the protrusions in the second section.

The spacing of the protrusions may vary along the length of the wire member. For example, the spacing of the protrusions in an edge portion of the wire member may be smaller than that in a central portion. Additionally, the mesh member may be configured such that the area of holes in an edge portion of the mesh member is larger than that in a central portion, thereby improving fixation and traction at the ends.

The end portions of the mesh member may be coupled by thermal fusion or bonding.

The mesh assembly may further include an insertion tube extending in a longitudinal direction, defining a channel space therein, and configured to receive the mesh member in the channel space. The insertion tube may include a portion compressed in a second direction perpendicular to the longitudinal direction. At the compressed portion, a cross-section of the channel space may have a reduced width in the second direction and an increased width in a third direction perpendicular to both the first and second directions.

These and other features and advantages of the present disclosure will become more apparent from the following detailed description of embodiments thereof. According to an embodiment of the present disclosure, a mesh member of a mesh assembly is configured in a planar shape or a cylindrical shape by a plurality of wire members crossing each other, thereby providing a mesh assembly with enhanced fixing force and pulling force for biological tissues, as well as excellent adhesion, retention, and fixation to skin tissue.

### BRIEF DESCRIPTION OF THE DRAWINGS

The above and other objectives, features and other advantages of the present disclosure will be more clearly understood from the following detailed description when taken in conjunction with the accompanying drawings, in which:

FIG. 1 is a view showing a portion of a mesh member 100 constituting a mesh assembly according to an embodiment of the present disclosure;

FIG. 2 is a view showing a portion of a mesh member constituting a mesh assembly according to another embodiment of the present disclosure;

FIG. 3 is a view showing a mesh assembly including a mesh member and a core 200 according to another embodiment of the present disclosure;

FIG. 4 is a view showing a mesh assembly including a mesh member and a core according to another embodiment of the present disclosure;

FIG. 5 is a view exemplifying the direction of protrusions formed on a wire member;

FIG. 6 is a view exemplifying the area of a hole formed in a mesh member;

FIG. 7 is a view exemplifying the spacing of protrusions formed on a wire member;

FIG. 8 is a view showing a portion of a mesh member constituting a mesh assembly according to another embodiment of the present disclosure;

FIGS. 9A and 9B are views showing various embodiments of a plurality of protrusions formed on a wire member;

FIG. 10 is a view exemplarily showing a portion of a mesh assembly according to the present disclosure;

FIGS. 11 and 12 are views exemplarily showing an insertion tube that can be applied to a mesh assembly according to the present disclosure;

FIGS. 13 and 14 are views exemplarily showing another insertion tube that can be applied to a mesh assembly according to the present disclosure; and

FIGS. 15 and 16 are views exemplarily showing another insertion tube 400C that can be applied to a mesh assembly 40 according to the present disclosure.

### DETAILED DESCRIPTION

The present disclosure may be modified in various ways and implemented by various exemplary embodiments, so that specific exemplary embodiments are shown in the drawings and will be described in detail herein. However, it is to be understood that the present disclosure is not limited to the specific exemplary embodiments, but includes all modifications, equivalents, and substitutions included in the spirit and the scope of the present disclosure. Similar reference numerals are assigned to similar components in the following description of drawings.

Unless defined otherwise, it is to be understood that all the terms used in the specification including technical and scientific terms have the same meanings as those that are understood by those who skilled in the art. It will be further understood that terms such as terms defined in common dictionaries should be interpreted as having a meaning that is consistent with their meaning in the context of this specification and the relevant art and will not be interpreted in an idealized or overly formal sense unless expressly so defined herein.

Hereinafter, exemplary embodiments of the present disclosure will be described in more detail with reference to the accompanying drawings.

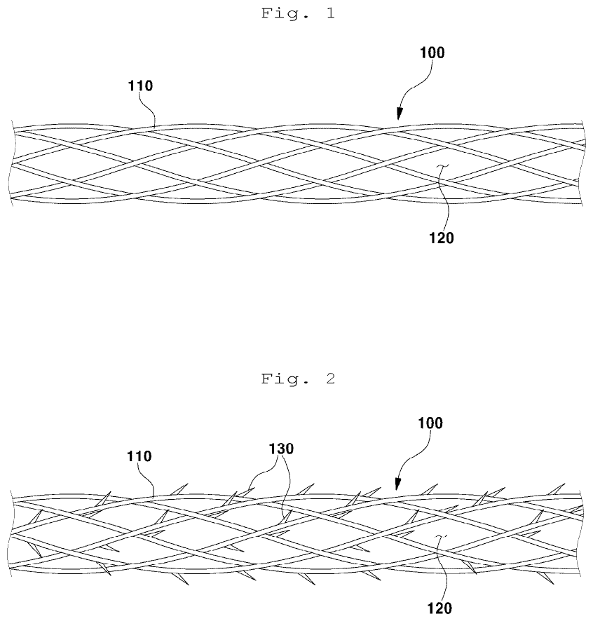

FIG. 1 is a view showing a portion of a mesh member 100 constituting a mesh assembly 10 according to an embodiment of the present disclosure. Though not shown in FIG. 1, an insertion tube such as a cannula or a needle may be provided at the front end of the mesh assembly 10 and the mesh member 100 may be coupled to or inserted in the insertion tube.

The mesh member 100 of the mesh assembly 10 can be inserted into the skin tissue of a human body through an insertion tube such as a cannula. Referring to FIG. 1, the mesh member 100 of the mesh assembly 10 may include a plurality of wire members 110 and may be formed into a cylindrical shape. Holes may be provided between the plurality of wire members 110.

The plurality of wire members 110 may extend helically along the longitudinal direction of the mesh member 100. The plurality of wire members 110 may form a 3-dimensional cylindrical structure by being interwoven with each other.

The mesh member 100 may form a 3-dimensional structure similar to a cylinder by forming a passage with a predetermined diameter therein. In the embodiment shown in FIG. 1, the passage with a predetermined diameter formed in the mesh member 100 is empty. That is, no other member is not disposed inside the mesh member 100. However, in another embodiment to be described below, another member (a core, etc.) may be disposed inside the mesh member 100.

The mesh member 100 may be made of a medical-grade material suitable for implantation or insertion in a human body. For example, the mesh member 100 may be made of a nonabsorbable polymer, a biodegradable polymer, or the like that is not harmful to the human body and is biocompatible.

Further, the mesh member 100 may be made of a hydrolyzable material. For example, the mesh member 100 may be made of a material containing polydioxanone (PDO). Alternatively, the mesh member 100 may be made of a material comprising one or more polymers selected from the group consisting of polylactic acid (PLA), poly-L-lactide (PLLA), polycaprolactone (PCL), polyhydroxyalkanoate (PHA), polyhydroxybutyrate (PHB), poly-D-lactide, and poly-DL-lactide (PDLLA).

In the embodiment shown in FIG. 1, the interior of the mesh member 100 of the mesh assembly 10 is hollow. According to this configuration, when the mesh member 100 is inserted into the skin, skin tissue can easily penetrate into the mesh member 100. Accordingly, integration of the mesh member 100 and skin tissue can be enhanced.

FIG. 2 is a view showing a portion of a mesh member 100 constituting a mesh assembly 10 according to another embodiment of the present disclosure.

Referring to FIG. 2, the mesh member 100 of the mesh assembly 10 may include a plurality of wire members 110 and may be formed in a cylindrical structure. Holes may be provided between the plurality of wire members 110.

The mesh member 100 of the mesh assembly 10 may be configured in a cylindrical shape to define a passage having a predetermined diameter therein. The passage defined inside the mesh assembly 10 including the mesh member 100 is hollow. The description of FIG. 1 may be referred to in relation to the mesh member 100.

The mesh member 100 may include a plurality of protrusions 130 protruding from the surface of the plurality of wire members 110. The protrusions 130 may secure the mesh member 100 in place within the skin tissue, or apply effective tension to pull the skin.

The plurality of protrusions 130 can generate tension by engaging tissues within the skin. The plurality of protrusions 130 may function to reduce or eliminate sagging or wrinkles of the skin. The plurality of protrusions 130 protrude from the surfaces of the plurality of wire members 110 and may be arranged at predetermined intervals.

The plurality of protrusions 130 can provide a secure engagement with skin tissues as the skin is pulled during the procedure. After the mesh member 100 is inserted into the skin using an insertion instrument and then the insertion instrument is removed, tension may be applied through a pulling force exerted by a practitioner.

The plurality of protrusions 130 may help maintain the elevated position of the mesh member 100 under tension. Further, the plurality of protrusions 130 may prevent the mesh member 100 from shifting downward after the procedure. That is, the plurality of protrusions 130 may be fixed to skin tissue during the procedure, thereby not only providing tension, but also preventing sagging of the skin in the long term.

The plurality of protrusions 130 may be formed regular or irregular intervals along the plurality of wire members 110. A three-dimensional cylindrical shape may be formed by spirally interweaving the plurality of wire members 110 having the plurality of protrusions 130 across each other. In the three-dimensional cylindrical mesh member 100, the plurality of protrusions 130 may extend in random directions. In another embodiment, a plurality of protrusions of the mesh member 100 may extend in a predetermined direction.

In the embodiment shown in FIG. 2, the interior of the mesh member 100 of the mesh assembly 10 is hollow. According to this configuration, when the mesh member 100 is inserted into the skin, skin tissue can easily grow into the mesh member 100. Accordingly, integration of the mesh member 100 and skin tissue can be facilitated.

Further, in the embodiment of FIG. 2, a plurality of protrusions 130 are formed on a plurality of wire members 110 of the mesh member 100. According to this configuration, a fixing force and a pulling force of the mesh member 100 with respect to the skin tissue may be enhanced.

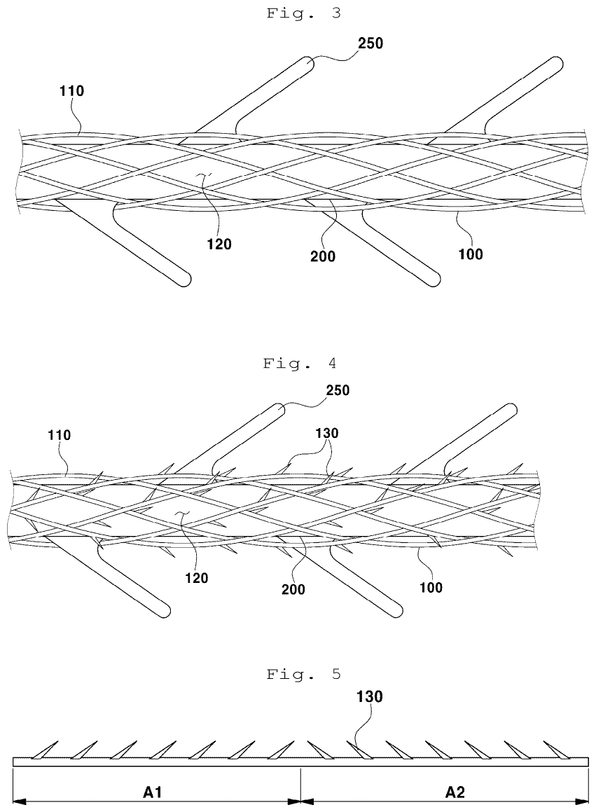

FIG. 3 is a view showing a mesh assembly 10 including a mesh member 100 and a core 200 according to another embodiment of the present disclosure.

Referring to FIG. 3, a mesh assembly 10 may include a mesh member 100 and a core 200. The core 200 may include a cog protruding from the surface.

The mesh member 100 of the mesh assembly 100 may include a plurality of wire members 110 and may be formed in a cylindrical shape. Holes may be provided between the plurality of wire members 110.

The mesh member 100 may be configured in a cylindrical shape forming a passage having a predetermined diameter therein. The core 200 extending in the longitudinal direction of the mesh member 100 may be disposed in the passage in the mesh assembly 10 including the mesh member 100.

The core 200 may be positioned in the mesh member 100. The core 200 may be formed with a diameter smaller than the diameter of the passage. The cross-section of the core 200 may be a circle. However, the cross-sectional shape of the core 200 is not limited to this embodiment and may be configured in various shapes such as an ellipse and a polygon.

The core 200 may include one or more cogs protruding and extending from the surface. The cog 250 may provide from the surface of the core 200 in an inclination direction toward a side. The cog 250 may be formed such that the end extends outside the mesh member 100. That is, the end of the cog 250 may protrude out of the mesh member 100 through a hole 120 between the plurality of wire members 110 included in the mesh member 100.

A plurality of cogs 250 may be formed on the upper portion and the lower portion of the core 200. That is, the plurality of cogs 250 may be formed alternately with spacing of 180 degrees on the outer circumferential surface of the core 200. However, the positions where the cogs 250 are formed are not limited to this embodiment, and, for example, the cogs 250 may be formed alternately with various spacing such as 90 degrees and 120 degrees on the outer circumferential surface of the core 200.

The core 200 and the mesh member 100 may be combined by thermal bonding (thermal fusion), an adhesive, or the like. That is, an end of the core 200 and an end of the mesh member 100 may be fused by heat or bonded by an adhesive, whereby the core 200 and the mesh member 100 can be coupled.

In the embodiment shown in FIG. 3, the core 200 is formed in the mesh member 100 of the mesh assembly 10 and cogs 150 protruding from the surface of the core 200 are formed on the core 200. According to this configuration, a fixing force and a pulling force of the mesh assembly 10 for skin tissues can be improved.

FIG. 4 is a view showing a mesh assembly 10 including a mesh member 100 and a core 200 according to another embodiment of the present disclosure.

Referring to FIG. 4, a mesh assembly 10 may include a mesh member 100 and a core 200. The mesh member 100 may include protrusions 130 protruding from the surface. The description about the protrusions 130 of the embodiment of FIG. 2 can be refereed to for the mesh member 100 and the protrusions 130.

The core 200 may include cogs 250 protruding from the surface. The description about the core 200 and the cogs 250 of the embodiment of FIG. 3 can be referred to for the core 200 and the cogs 250.

In the embodiment shown in FIG. 4, the core 200 is formed in the mesh member 100 of the mesh assembly 10, the cogs 150 protruding from the surface of the core 200 are formed on the core 200, and protrusions 130 protruding from the surface of the mesh member 100 of the mesh assembly 10 are formed on the mesh member 100. According to this configuration, a fixing force and a pulling force of the mesh assembly 10 for skin tissues can be further improved.

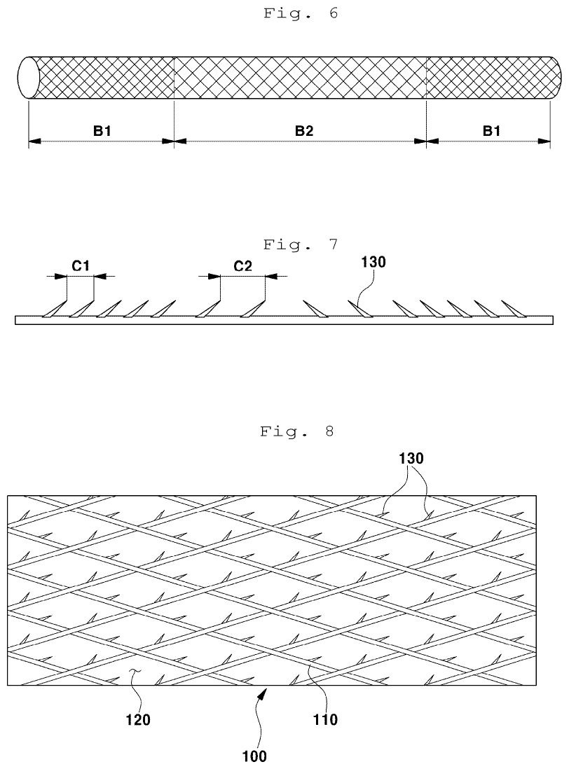

FIG. 5 is a view illustrating exemplary directions of the protrusions 130 formed on a wire member 110. The wire member 110 having a plurality of protrusions 130 is used in the manufacture of the mesh member 100. A mesh member 100 having a three-dimensional cylindrical shape may be formed by interweaving a plurality of the wire members 110 shown in FIG. 5.

Referring to FIG. 5, the wire member 110 may include a plurality of protrusions 130 protruding from its surface. The wire member 110 may include a first section A1 and a second section A2 having different extension directions of the protrusions.

The extension direction of the plurality of protrusions disposed in the first section A1 of the wire member 110 may be different from the extension direction of the plurality of protrusions disposed in second section A2 of the wire member 110.

In an embodiment of the present disclosure, as shown in FIG. 5, the extension direction of the plurality of protrusions 130 disposed in the first section A1 of the wire member 110 may be opposite to the extension direction of the plurality of protrusions disposed in second section A2 of the wire member 110. When a mesh member 100 having a cylindrical shape is formed by interweaving a plurality of such wire members 110, the protrusions 130 in the first sections A1 may be oriented in a direction generally opposite to that of the protrusions in the second sections A2.

As in the embodiment shown in FIG. 5, by configuring the plurality of protrusions 130 disposed in the first section A1 to be oriented opposite to those in the second section A2, the fixing force and the pulling force of the mesh assembly 10 can be further enhanced.

FIG. 6 is a view illustrating the area of the holes 120 formed on the mesh member 100. In an embodiment, the mesh member 110 may include a plurality of sections, such as B1 and B2, having different hole areas.

Referring to FIG. 6, the mesh member 100 may include a center section B2 and edge sections B1. In an embodiment, the area of the plurality of holes in the center section B2 of the mesh member 100 may be larger than the area of the plurality of holes in the edge sections B1 of the mesh member 100.

However, the present disclosure is not limited to the embodiment of FIG. 6. In another embodiment, the area of the plurality of holes in the center section B2 of the mesh member 100 may be smaller than the area of the plurality of holes in the edge sections B1 of the mesh member 100.

Further, though not shown in FIG. 6, protrusions 130 protruding from the surface of the mesh member 100 may be formed on the mesh member 100 and the area of the holes may vary depending on the number and spacing of the protrusions 130 formed on the mesh member 100.

As in the embodiment shown in FIG. 6, the mesh member 100 may be configured to include a plurality of sections, such as B1 and B2, having different hole areas, thereby allowing the fixing and pulling forces for skin tissues to vary by section.

FIG. 7 is a view exemplifying the spacing of the protrusions 130 formed on a wire member 110.

Referring to FIG. 7, the wire member 110 may include a plurality of protrusions 130 protruding from the surface. In an embodiment, the wire member 110 may be configured such that the spacing C1 of a plurality of protrusions 130 disposed at the edge portions of the wire member 110 may be smaller than the spacing C2 of a plurality of protrusions 130 disposed at the center portion of the wire member 100. When a mesh member 100 having a cylindrical shape is manufactured by interweaving a plurality of wire members 110 of this embodiment, the spacing C1 of the plurality of protrusions 130 disposed at the edge portions of the mesh member 100 is smaller than the spacing C2 of the plurality of protrusions 130 disposed at the center portion of the mesh member 100.

When the spacing of the protrusions 130 are uniform, the fixing force and the pulling force for skin tissue at both end portions of the mesh member 100 may be relatively lower than those at the center portion of the mesh member 100. According to the embodiment shown in FIG. 7, the spacing of the plurality of protrusions 130 at the edge portions is smaller than the pacing of the plurality of protrusions 130 at the center portion, thereby enhancing the fixing and pulling forces for skin tissue at both ends of the mesh member 100.

In an embodiment, the hole area of meshes in a section where the spacing of the protrusions 130 is smaller may be larger than that in a section where the spacing is greater. In the section with narrower spacing, the plurality of protrusions 130 may interfere with each other. By configuring the mesh to have a larger hole area in such sections, interference between the protrusions 130 can be reduced.

FIG. 8 is a view showing a portion of a mesh member 100 constituting a mesh assembly 10 according to another embodiment of the present disclosure.

Referring to FIG. 8, the mesh member 100 of the mesh assembly 100 may include a plurality of wire members 110 crossing each other. The mesh member 100 may be configured in a plane shape having a predetermined area. The plurality of wire members 110 may include protrusions 130 protruding from the surface.

Unless contradicted as description about the mesh member 100, the wire members 110, and the protrusions 130 according to the embodiment of FIG. 8, the description about the mesh member 100, the wire members 110, and the protrusions 130 shown in FIGS. 1 to 7 can be referred to. The description about the extension direction of the protrusions 130 shown in FIG. 5, the hole area of the mesh member 100 shown in FIG. 6, and the spacing of the protrusions 130 shown in FIG. 7 can be applied to the mesh member 100, the wire members 110, and the protrusions 130 according to the embodiment of FIG. 8.

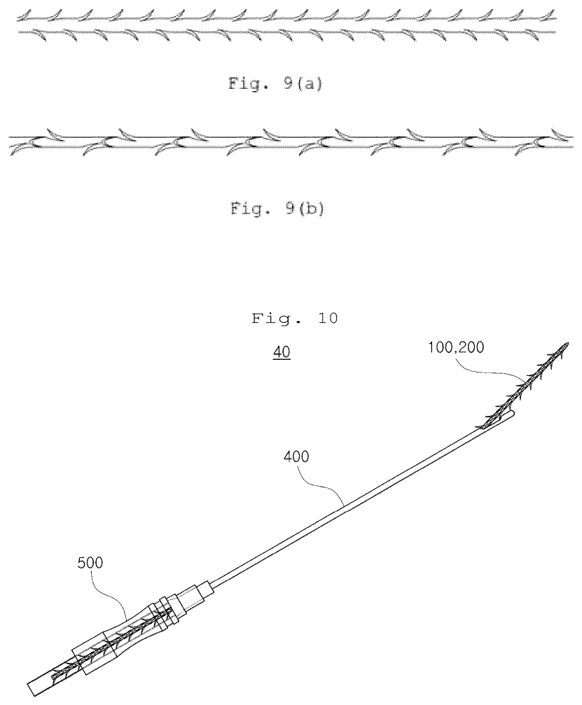

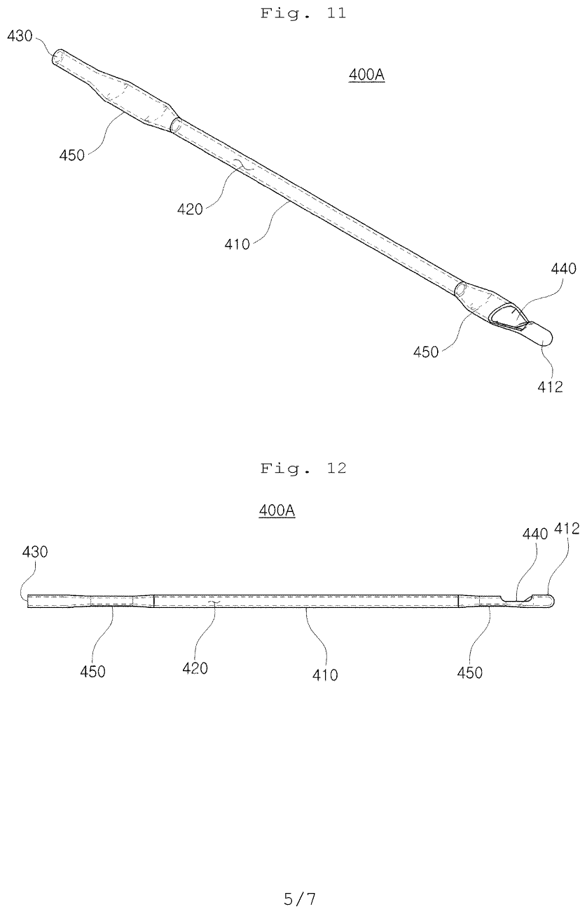

FIG. 9A and 9B are views showing various embodiments of a plurality of protrusions 130 formed on a wire member 110.

FIG. 9A exemplifies a plurality of protrusions 130 arranged with spacing of 180 degrees in the circumferential direction of the wire member 110. Referring to FIG. 9A, a plurality of protrusions 130 may be longitudinally arranged. The plurality of protrusions 130 may be arranged alternately with spacing of 180 degrees in the circumferential direction.

FIG. 9B exemplifies a plurality of protrusions 130 arranged with spacing of 90 degrees in the circumferential direction of the wire member 110. Referring to FIG. 9B, a plurality of protrusions 130 may be longitudinally arranged. The plurality of protrusions 130 may be arranged alternately with spacing of 90 degrees in the circumferential direction.

Assuming that a protrusion 131 disposed on the lower portion of the wire member 110 is a first protrusion, a second protrusion 132 may be formed ahead with a spacing of 90 degrees in the circumferential direction with respect to the first protrusion. A third protrusion 133 may be formed over with a spacing of 90 degrees with respect to the second protrusion 132. A fourth protrusion is disposed behind and is not shown in the figures, but may be disposed behind with a spacing of 90 degrees with respect to the third protrusion 133.

The shapes of the protrusions 130 shown in FIG. 2, FIG. 5, FIG. 9A, and FIG. 9B only exemplify the shape of protrusions 130 of wire members 110 according to the present disclosure and the present disclosure is not limited to this example.

FIG. 10 is a view exemplarily showing a portion of a mesh assembly 40 according to the present disclosure.

Referring to FIG. 10, the mesh assembly 40 may further include an insertion tube 400 in the mesh assembly 10 relating to the embodiment described above. That is, the mesh assembly 40 may include a mesh member 100, a core 200, and an insertion tube 400. The mesh assembly 40 according to the present disclosure is not limited to the embodiment shown in FIG. 10 and may include a mesh member 100 and an insertion tube 400 without a core 200.

Assuming the extension direction of the core 200 is a first direction, the insertion tube 400 may extend in the first direction. The insertion tube 400 may include an internal channel space 420. In the present disclosure, the insertion tube 400 includes a cannula, Stewart needles, skin needles, catheter needles, injection needles, etc., and is a term including all kinds of medical instruments having a channel space 420 therein.

The channel space 420 may extend from an opening 430 positioned at the rear end of the insertion tube 400 to an opening 440 positioned at the front end of the insertion tube 400. As shown in FIG. 10, the mesh member 100 and the core 200 can be inserted into the channel space 420 of the insertion tube 400. The mesh member 100 and the core 200 is put into the opening 430 at the rear end of the channel space 420 and may be inserted to the opening 440 at the front end or beyond the opening 440 to pass through the opening 440. A connector 500 may be provided at the rear end of the insertion tube 400 for the convenience of use.

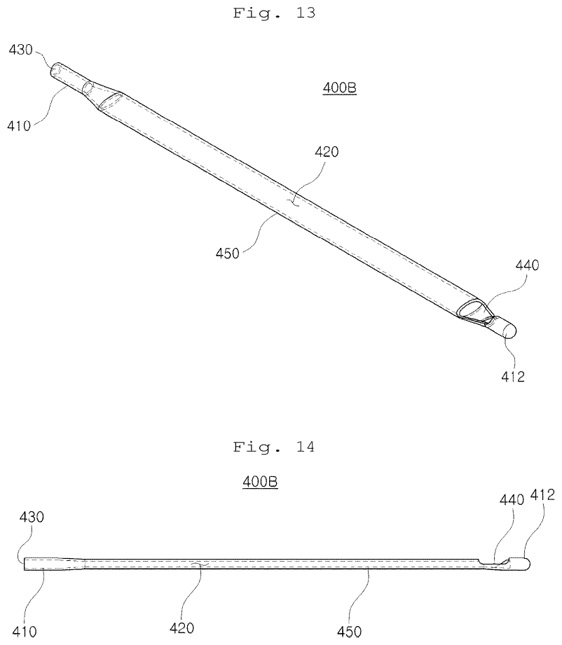

FIGS. 11 and 12 are views exemplarily showing an insertion tube 400A that can be applied to the mesh assembly 40 according to the present disclosure.

According to an embodiment of the present disclosure, the insertion tube 400A may include a compressed portion 450. In this case, the term ‘compressed portion 450’ is only for describing the shape of the insertion tube 400A and is not limited to being formed by compressing a main body 410 of the insertion tube 400A.

Assuming that the extension direction of the insertion tube 420 is a first direction, a direction perpendicular to the first direction is a second direction, and a direction perpendicular to both of the first direction and the second direction is a third direction, the main body 410 of the insertion tube 400 is compressed in the second direction at the compressed portion 450, whereby the cross-section of the compressed portion 450 may have a reduced width in the second direction. The compressed portion 450 may have an increased width in the third direction.

In the mesh assembly 40 relating to an embodiment of the present disclosure, since cogs 250 protrude from the core 200, a cross-section having a cog 250 of the core 200 is larger in width in a specific direction (i.e., the third direction) than another direction (i.e., the second direction). If the channel space 420 of the insertion tube 400 maintains a circular cross-section and has an increased inner diameter, and accordingly, when the mesh member 100 and the core 200 are accommodated therein, there is a possibility that the diameter of the entire insertion tube 400 becomes excessively large. In this case, there is a possibility that a hole having an increased size is formed at a procedure part of a patient and accordingly a scar is left.

When the compressed portion 450 is formed at the insertion tube 400, as in an embodiment of the present disclosure, the cross-section of the channel space 420 has a reduced width in the second direction and an increased with in the third direction. This insertion tube 400 is suitable for accommodating a core 200 having a small width in the second direction and a large width in the third direction. In an embodiment of the present disclosure, it is possible to substantially form the compressed portion 450 by compressing a portion of a cannula of a basic shape, for example, a portion of a cannula of 18 gauges.

In the example shown in FIGS. 11 and 12, a plurality of compressed portions 450 is formed at the insertion tube 400. The position and the number of the compressed portion 450 may be changed, if necessary. The front end 412 of the insertion tube 400 is rounded, so it may have a smooth curved surface without a sharp corner. The front opening 440 of the channel space 420 may be formed close to the front end 412.

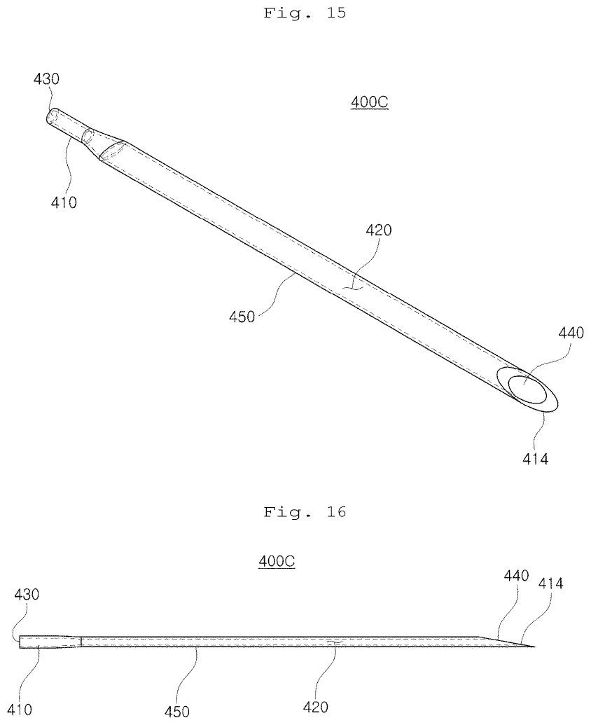

FIGS. 13 and 14 are views exemplarily showing another insertion tube 400B that can be applied to the mesh assembly 40 according to the present disclosure.

In the insertion tube 400B shown in FIGS. 13 and 14, a compressed portion 450 is formed at a most part of the main body 410 of the insertion tube 400 and extends to the front opening 440. In the insertion tube 400B shown in FIGS. 13 and 14, the rear end of the main body 410 of the insertion tube 400 maintains a non-compressed state, but, in another embodiment, the compressed portion 450 may be formed throughout the entire main body 410 of the insertion tube 400 including the rear end of the main body 410.

FIGS. 15 and 16 are views exemplarily showing another insertion tube 400C that can be applied to the mesh assembly 40 according to the present disclosure. In the insertion tube 400C shown in FIGS. 15 and 16, the front end 414 of the insertion tube 400 is formed in a shape having a cut surface and the front opening 440 of the channel space 420 is formed at the cut surface of the front end 414. This structure may have the advantage that the compressed portion 450 can extend to the front end 414.

The scope of the present disclosure is defined by the following claims, and all of changes and modifications obtained from the meaning and range of claims and equivalent concepts should be construed as being included in the scope of the present disclosure.

What is claimed is:

1. A mesh assembly that is inserted in skin tissue of a human body, the mesh assembly comprising a mesh member having a plurality of wire members crossing each other,
wherein the mesh member is configured in a cylindrical shape to define a passage having a predetermined diameter therein,
one or more of the plurality of wire members of the mesh member includes at least one protrusion extending from a surface thereof, and
the entire passage defined inside the mesh member is an empty space in which no other material is disposed.

2. The mesh assembly of claim 1,
wherein an extension direction of a plurality of protrusions in a first section of the wire member differs from an extension direction of a plurality of protrusions in a second section of the wire member.

3. The mesh assembly of claim 1, wherein an extension direction of a plurality of protrusions in a first section of the wire member is opposite to the extension direction of a plurality of protrusions in a second section of the wire member.

4. The mesh assembly of claim 1, wherein the spacing of a plurality of protrusions disposed in an edge portion of the wire member is smaller than the spacing of a plurality of protrusions disposed at a central portion of the wire member.

5. The mesh assembly of claim 1, wherein the spacing of a plurality of protrusions disposed in an edge portion of the wire member is smaller than the spacing of a plurality of protrusions disposed at a central portion of the wire member, and
areas of a plurality of holes at an edge portion of the mesh member corresponding to the edge portion of the wire member are larger than areas of a plurality of holes at a central portion of the mesh member corresponding to the central portion of the wire member.

6. The mesh assembly of claim 1, wherein end portions of the mesh member are coupled by thermal fusion or bonding.

7. The mesh assembly of claim 1, further comprising an insertion tube extending in a first direction that is a longitudinal direction, having a channel space formed therein, and configured to accommodate the mesh member in the channel space.

8. The mesh assembly of claim 7, wherein the insertion tube includes a portion compressed in a second direction perpendicular to the first direction, and a cross-section of the channel space at the compressed portion has a reduced width in the second direction and has an increased width in a third direction perpendicular to both the first and the second direction.
